(19) United States
(12) Patent Application Publication
(10) Pub. No.: US 2020/0107880 A1
(43) Pub. Date: Apr. 9, 2020

(54) SURGICAL INSTRUMENTS AND METHODS FOR HEPATIC-RELATED SURGICAL PROCEDURES

(71) Applicant: COVIDIEN LP, Mansfield, MA (US)

(72) Inventors: Thomas E. Drochner, Longmont, CO (US); James D. Allen, IV, Broomfield, CO (US); John A. Hammerland, III, Arvada, CO (US); Daniel A. Joseph, Golden, CO (US); Rachael L. Kabala, Fort Collins, CO (US)

(21) Appl. No.: 16/580,862

(22) Filed: Sep. 24, 2019

Related U.S. Application Data
(60) Provisional application No. 62/740,624, filed on Oct. 3, 2018.

(51) Int. Cl. A61B 18/18; A61B 34/35

(52) U.S. Cl. CPC A61B 18/1815; A61B 2018/00589; A61B 34/35

(57) ABSTRACT

A method of treating liver tissue includes progressively grasping liver tissue, applying a first energy to the grasped liver tissue during the progressive grasping to permanently close parenchyma of the liver tissue, fully grasping the liver tissue, and applying a second energy to the fully grasped liver tissue to permanently close large vessels of the fully grasped liver tissue.

### CROSS REFERENCE TO RELATED APPLICATION

The present application claims the benefit of and priority to U.S. Provisional Application Ser. No. 62/740,624, filed on Oct. 3, 2018 the entire contents of which are incorporated herein by reference.

### BACKGROUND

### Technical Field

The present disclosure relates generally to surgical instruments and methods. More specifically, the present disclosure relates to surgical instruments and methods for treating, e.g., coagulating, sealing, and/or transecting, tissue in hepatic-related surgical procedures.

### Background of Related Art

Surgical instruments and methods for energy-based tissue treatment utilize mechanical clamping action and application of energy, e.g., electrosurgical energy, ultrasonic energy, microwave energy, light energy, etc., to affect hemostasis by heating tissue to coagulate, cauterize, and/or seal tissue. Coagulation may be sufficient to achieve hemostasis on non-vascular tissues, small blood vessels, e.g., vessels below about two millimeters in diameter, and tissues including small vessels. With respect to larger blood vessels, e.g., vessels above about two millimeters in diameter, and tissues including larger vessels, coagulation may be insufficient to achieve hemostasis; instead, these larger vessels and tissues including the same may be required to be sealed, a process by which the collagen in the tissue is heated up, denatured, and reformed into a fused mass to permanently close the vessel(s). Once hemostasis is achieved, e.g., via coagulation (for smaller vessels) or sealing (for larger vessels), the tissue may be cut (mechanically, electrically, or electro-mechanically) to divide the tissue.

Hepatic resection is a surgical procedure with many challenges due to an increased risk of bleeding and complications relating to the anatomy of the liver. Currently, surgeons utilize a combination of different instruments and techniques to perform the various different tasks associated with a hepatic resection.

### SUMMARY

As used herein, the term “distal” refers to the portion of the instrument or component thereof that is being described that is further from a user, while the term “proximal” refers to the portion of the instrument or component thereof that is being described that is closer to a user. Further, to the extent consistent, any of the aspects described herein may be used in conjunction with any of the other aspects described herein.

Provided in accordance with aspects of the present disclosure is a method of treating liver tissue including progressively grasping liver tissue, applying a first energy to the grasped liver tissue during the progressive grasping to permanently close parenchyma of the liver tissue, fully grasping the liver tissue, and applying a second energy to the fully grasped liver tissue to permanently close large vessels of the fully grasped liver tissue.

In an aspect of the present disclosure, applying the first energy includes coagulating the parenchyma to permanently close the parenchyma.

In another aspect of the present disclosure, the first energy is standard bipolar energy.

In another aspect of the present disclosure, applying the second energy includes sealing the vessels to permanently close the vessels.

In still another aspect of the present disclosure, the second energy is a vessel-sealing energy.

In yet another aspect of the present disclosure, fully grasping the liver tissue includes grasping the liver tissue with vessel-sealing forces and/or grasping the liver tissue between tissue-treating surfaces defining vessel-sealing gap distances therebetween.

In still yet another aspect of the present disclosure, applying the first energy includes one of: continuously applying the first energy during the progressive grasping, intermittently applying the first energy during the progressive grasping, or applying the first energy at one or more stop points during the progressive grasping.

In another aspect of the present disclosure, the method further includes cutting the liver tissue.

Another method of treating liver tissue provided in accordance with aspects of the present disclosure includes progressively approximating first and second jaw members to grasp liver tissue between respective first and second tissue-treating surfaces of the first and second jaw members, conducting a first energy between the first and second tissue-treating surfaces and through the grasped liver tissue to permanently close parenchyma of the grasped living tissue as the first and second jaw members are progressively approximated, further approximating the first and second jaw members to fully grasp the liver tissue between the first and second tissue-treating surfaces, and conducting a second energy between the first and second tissue-treating surfaces and through the fully grasped liver tissue to permanently close large vessels of the fully grasped liver tissue.

In an aspect of the present disclosure, conducting the first energy includes coagulating the parenchyma to permanently close the parenchyma.

In another aspect of the present disclosure, the first energy is standard bipolar energy.

In another aspect of the present disclosure, conducting the second energy includes sealing the vessels to permanently close the vessels.

In still another aspect of the present disclosure, the second energy is a vessel-sealing energy.

In yet another aspect of the present disclosure, fully grasping the liver tissue includes grasping the liver tissue with vessel-sealing forces and/or grasping the liver tissue with vessel-sealing gap distances defined between the tissue-treating surfaces.

In still yet another aspect of the present disclosure, conducting the first energy includes one of: continuously conducting the first energy during the progressive grasping, intermittently conducting the first energy during the progressive grasping, or conducting the first energy at one or more stop points during the progressive grasping.

In another aspect of the present disclosure, the method further includes advancing a knife between the first and second jaw members to cut the liver tissue.

### BRIEF DESCRIPTION OF THE DRAWINGS

Various aspects and features of the present disclosure are described herein with reference to the drawings, wherein like reference numerals identify similar or identical components, and wherein:

FIG. 1 is a perspective view of a surgical instrument provided in accordance with the present disclosure;

FIG. 2 is a perspective view of another surgical instrument provided in accordance with the present disclosure;

FIG. 3 is a schematic illustration of a surgical system provided in accordance with the present disclosure;

FIG. 4A is a longitudinal, cross-sectional view of an end effector assembly of the surgical instrument of FIG. 1, wherein jaw members of the end effector assembly are disposed in a spaced-apart position;

FIG. 4B is a longitudinal, cross-sectional view of the end effector assembly of FIG. 4A, wherein the jaw members are disposed in an approximated position;

FIG. 5A is a perspective view of a distal portion of another surgical instrument provided in accordance with the present disclosure;

FIG. 5B is a side view of the distal portion of the surgical instrument of FIG. 5A;

FIG. 5C is a perspective view of the energy-applying jaw member of the surgical instrument of FIG. 5A;

FIGS. 6A-6D are progressive, representative illustrations of treatment of liver tissue in accordance with the present disclosure; and

FIG. 7 is a flow diagram illustrating a method of treating liver tissue in accordance with the present disclosure.

### DETAILED DESCRIPTION

The present disclosure provides surgical instruments and methods for treating, e.g., coagulating, sealing, and/or transecting, liver tissue in hepatic-related surgical procedures. Although the surgical instruments and methods of the present disclosure are detailed herein in use with respect to one another for treating liver tissue, it is contemplated that the surgical instruments and/or methods of the present disclosure may be used independently of one another for hepatic-related or other surgical procedures.

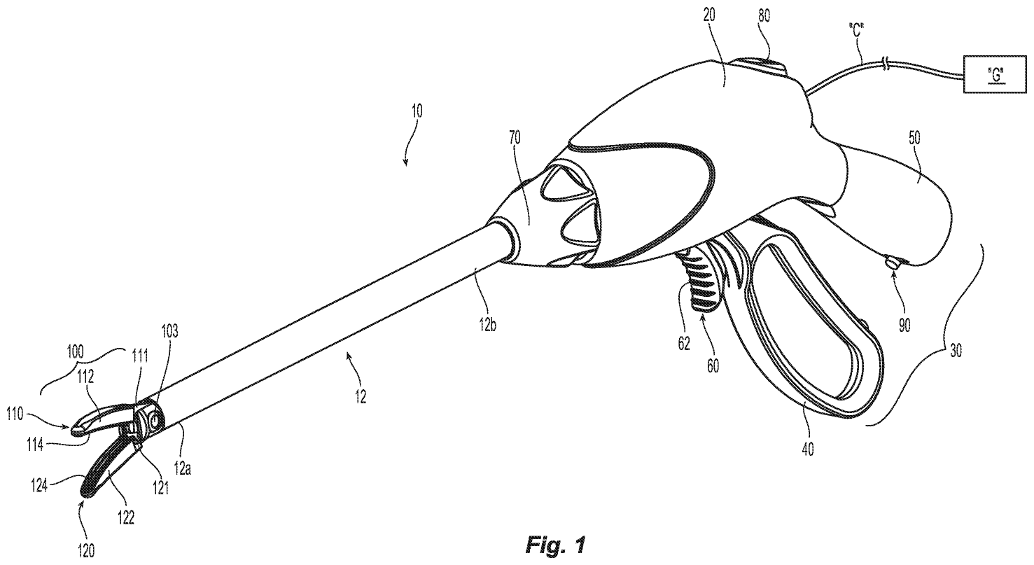

Turning to FIG. 1, a surgical instrument provided in accordance with the present disclosure is shown generally identified by reference numeral 10. Instrument 10 includes a housing 20, a handle assembly 30, a trigger assembly 60, a rotating assembly 70, first and second activation switches 80, 90, respectively, and an end effector assembly 100. Instrument 10 further includes a shaft 12 having a distal portion 12a configured to mechanically couple with end effector assembly 100 and a proximal portion 12b that mechanically couples with housing 20. Instrument 10 also includes cable “C” that connects instrument 10 to an energy source, e.g., an electrosurgical generator “G,” although instrument 10 may alternatively be configured as a battery-powered device. Cable “C” includes wires (not shown) extending therethrough, one or more of which has sufficient length to extend through shaft 12 in order to provide energy to one or both tissue-treating surfaces 114, 124 of jaw members 110, 120, respectively, of end effector assembly 100. First and second activation switches 80, 90, respectively, are coupled to tissue-treating surfaces 114, 124 and the electrosurgical generator “G” for selectively activating the electrosurgical generator “G” to supply first and second energies, respectively, to jaw members 110, 120 for treating tissue grasped therebetween, as detailed below.

The electrosurgical generator “G,” in embodiments, may be configured to supply a standard bipolar energy upon activation of first activation switch 80 and a vessel-sealing energy upon activation of second activation switch 90. The standard bipolar energy is configured to effect coagulation to permanently close non-vascular tissues, small blood vessels, and tissues including small vessels. The vessel-sealing energy is an advanced bipolar energy that may include an energy-delivery algorithm with varying energy delivery, feedback-based control, and/or other features to facilitate vessel-sealing as opposed to simply coagulating tissue. The vessel-sealing energy is suitable for permanently closing larger blood vessels and tissues including larger vessels. Vessel-sealing energies are detailed, for example and without limitation, in U.S. Pat. Nos. 7,303,557, 7,972,328, and 8,216,223, the entire contents of each of which is hereby incorporate herein by reference.

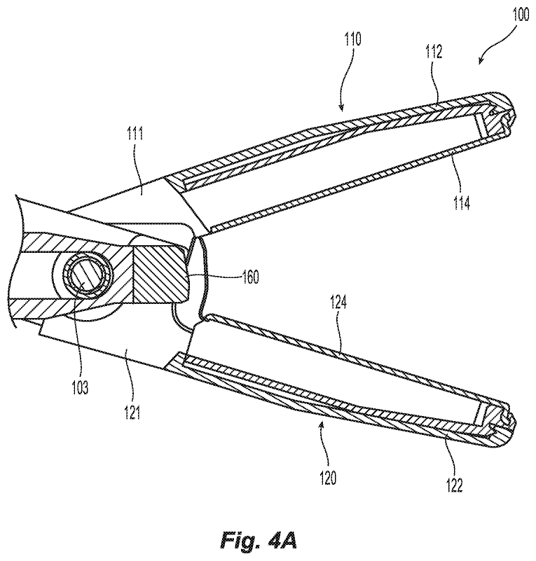

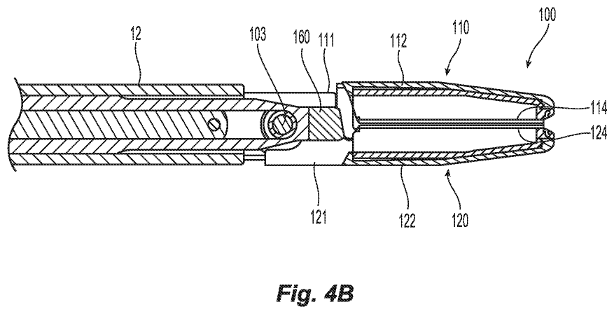

Handle assembly 30 of instrument 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both jaw members 110, 120 of end effector assembly 100 about a pivot 103 between a spaced-apart position (FIGS. 1 and 4A) and an approximated position (FIG. 4B) to grasp tissue between jaw members 110, 120. Movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 4B). Second activation switch 90 is disposed in-line with movable handle 40 such that second activation switch 90 is activated upon sufficient approximation of movable handle 40 relative to fixed handle 50. In embodiments, movable handle 40 is movable further towards fixed handle 50 from the depressed position to a further-depressed position to activate second activation switch 90. First activation switch 80 is a handswitch.

Trigger assembly 60 includes a trigger 62 coupled to housing 20 and movable relative thereto between an un-actuated position and an actuated position. Trigger 62 is operably coupled to a cutting mechanism including a knife 160 (FIGS. 4A and 4B) that is selectively advanceable between jaw members 110, 120 (and, in embodiments, through knife channels defined therein) to cut tissue grasped between jaw members 110, 120 of end effector assembly 100 upon actuation of trigger 62.

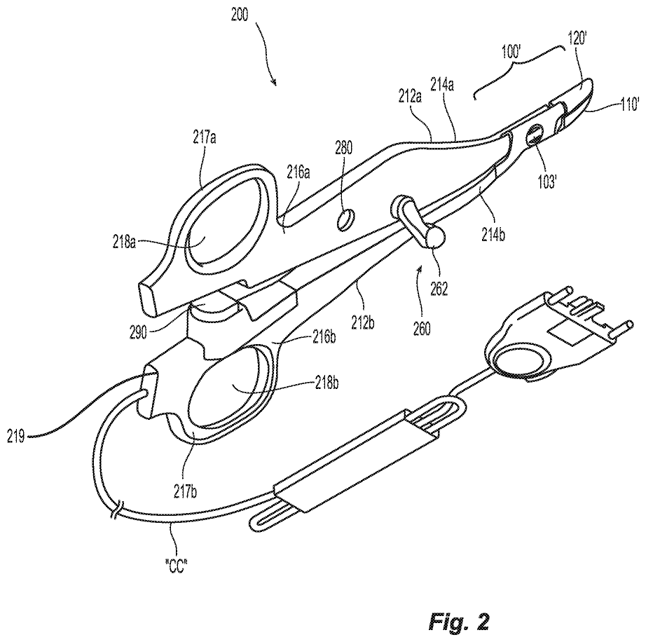

Referring to FIG. 2, another surgical instrument provided in accordance with the present disclosure is shown generally identified by reference numeral 200. Instrument 200 includes two elongated shaft members 212a, 212b, each having a proximal portion 216a, 216b, and a distal portion 214a, 214b, respectively. Instrument 200 is configured for use with an end effector assembly 100′ similar to end effector assembly 100 (FIGS. 1, 4A, and 4B). More specifically, end effector assembly 100′ includes first and second jaw members 110′, 120′ attached to respective distal portions 214a, 214b of shaft members 212a, 212b. Jaw members 110′, 120′ are pivotably connected about a pivot 103′. Each shaft member 212a, 212b includes a handle 217a, 217b disposed at the proximal portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another from an open position to a closed position to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of instrument 200, e.g., shaft member 212b, includes a proximal shaft connector 219 configured to connect forceps 210 to a source of energy, e.g., electrosurgical generator “G” (FIG. 1). Proximal shaft connector 219 secures a cable “CC” to instrument 200 that houses wires (not shown) electrically communicating with such that the user may selectively supply energy to jaw members 110′, 120′ for treating tissue and for energy-based tissue cutting.

First and second activation switches 280, 290, respectively, of instrument 200 are coupled to tissue-treating surfaces 114′, 124′ of jaw members 110′, 120′ and the electrosurgical generator “G” for selectively activating the electrosurgical generator “G” to supply first and second energies, respectively, to jaw members 110′, 120′ for treating tissue grasped therebetween, similarly as detailed above with respect to the electrosurgical generator “G” and first and second activation switches 80, 90 of surgical instrument 10 (see FIG. 1). Second activation switch 290 is an in-line switch disposed on shaft member 212b and configured to be actuated by shaft member 212a upon sufficient approximation of shaft members 212a, 212b. In embodiments, shaft members 212a, 212b are approximated relative to one another beyond the closed position to a further closed position to activate second activation switch 290. First activation switch 280 is a handswitch and may be disposed on shaft member 212a or shaft member 212b.

Instrument 200 further includes a trigger assembly 260 including a trigger 262 coupled to one of the shaft members, e.g., shaft member 212a, and movable relative thereto between an un-actuated position and an actuated position. Trigger 262 is operably coupled to a cutting mechanism including a knife (not shown, similar to knife 160 (FIGS. 4A-4B)) that is selectively advanceable between jaw members 110′, 120′ to cut tissue grasped therebetween, similarly as with jaw members 110, 120 of end effector assembly 100 (FIGS. 1, 4A, and 4B).

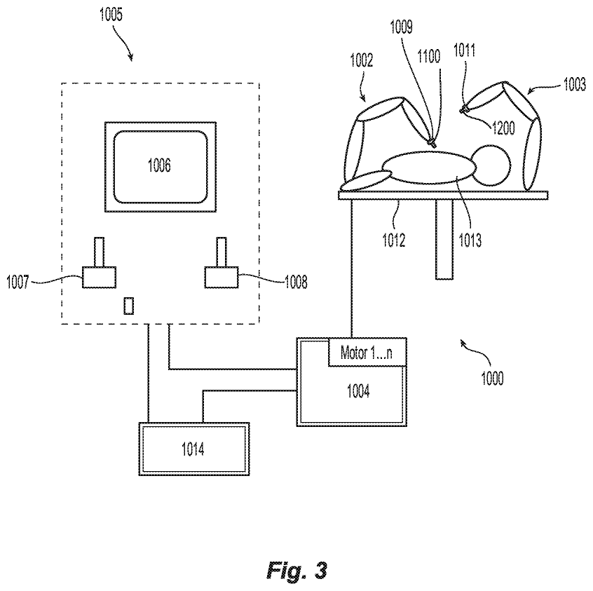

Referring to FIG. 3, a robotic surgical system exemplifying the aspects and features of the present disclosure is shown generally identified by reference numeral 1000. Robotic surgical system 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, allowing a surgeon to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assemblies 100, 100′ (FIGS. 1 and 2, respectively), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular via a computer program, in such a way that robot arms 1002, 1003, their attaching devices 1009, 1011, and/or end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Robotic surgical system 1000 is configured to couple to or incorporate an electrosurgical generator “G” (FIG. 1) that, in turn, is configured to supply first and second energies to tissue via end effector assembly 1100, similarly as detailed above with respect to instruments 100 (FIG. 1) and 200 (FIG. 2).

Turning to FIGS. 4A and 4B, end effector assembly 100 of surgical instrument 100 (FIG. 1) is detailed, keeping in mind that end effector assemblies 100′, 1100 (FIGS. 2 and 3, respectively) include similar features. End effector assembly 100, as noted above, includes first and second jaw members 110, 120. Each jaw member 110, 120 includes a proximal flange portion 111, 121, a distal body portion 112, 122 extending distally from the respective proximal portion 111, 121, and a tissue-treating surface 114, 124, respectively, supported on the respective distal body portion 112, 122. Proximal flange portions 111, 121 are pivotably coupled to one another about pivot 103 for moving one or both of jaw members 110, 120 between the spaced-apart and approximated positions, although other suitable mechanisms for pivoting jaw members 110, 120 relative to one another are also contemplated.

Distal body portions 112, 122 of jaw members 110, 120 support and retain tissue-treating surfaces 114, 124 on respective jaw members 110, 120 in opposed relation relative to one another. Tissue-treating surfaces 114, 124 are formed from an electrically conductive material, e.g., for conducting electrical energy therebetween for treating tissue, although tissue-treating surfaces 114, 124 may alternatively be configured to conduct any suitable energy, e.g., thermal, microwave, light, ultrasonic, etc., through tissue grasped therebetween for energy-based tissue treatment. As mentioned above, tissue-treating surfaces 114, 124 are coupled to activation switches 80, 90 and the electrosurgical generator “G” (see FIG. 1) such that energy may be selectively supplied to tissue-treating surface 114 and/or tissue-treating surface 124 and conducted therebetween and through tissue disposed between jaw members 110, 120 to treat tissue.

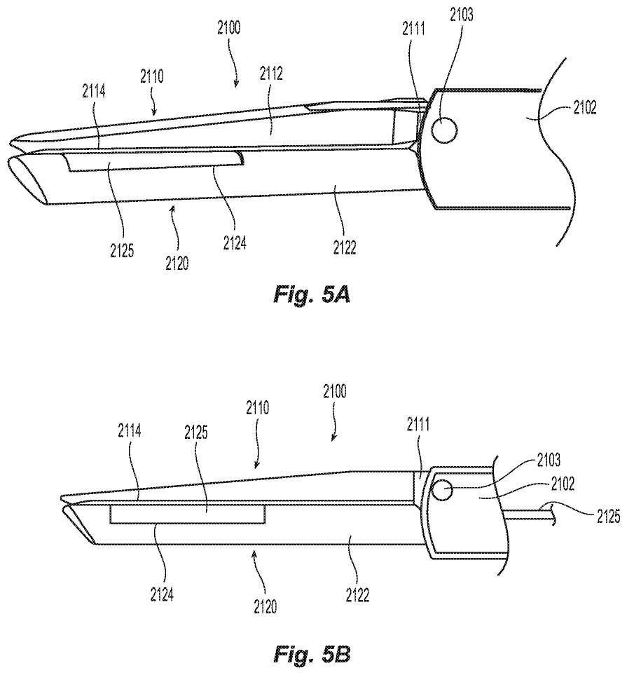

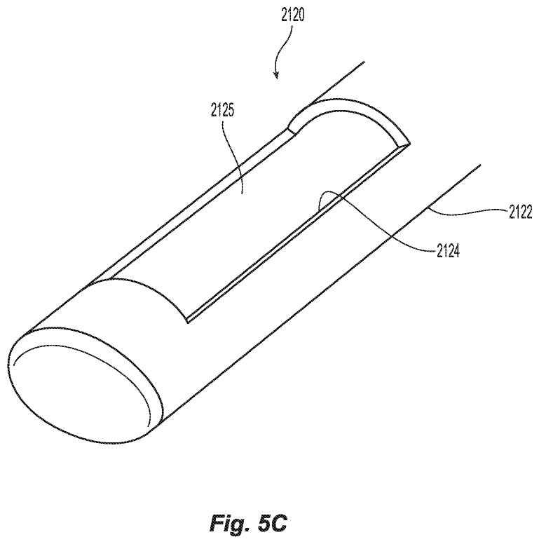

With reference to FIGS. 5A-5C, another end effector assembly provided in accordance with the present disclosure is shown generally identified by reference numeral 2100. End effector assembly 2100 may be configured for use with surgical instrument 10 (FIG. 1), surgical instrument 200 (FIG. 2), surgical system 1000 (FIG. 3), or any other suitable surgical instrument and/or system.

End effector assembly 2100 includes first and second jaw members 2110, 2120. One of the jaw members, e.g., jaw member 2110, functions as a movable clamping jaw, and includes a proximal flange portion 2111 pivotably coupled to jaw member 2120 and an outer shaft 2102 (or other suitable feature of the surgical instrument associated with end effector assembly 2100) about a pivot 2103 and a distal body portion 2112 extending distally from proximal flange portion 2111. Distal body portion 2112 defines a tissue-treating surface 2114. Jaw member 2110 is pivotable about pivot 2103 and relative to jaw member 2120 between a spaced-apart position and an approximated position for grasping tissue therebetween.

Jaw member 2120 includes a body 2122 extending through shaft 2102 and proximal flange portion 2111 of jaw member 2110 such that a distal portion of body 2122 opposes distal body portion 2112 of jaw member 2110. Body 2122 defines a cylindrical configuration (although other configurations are also contemplated), is formed from or includes suitable material to inhibit passage of microwave energy therethrough, and defines a distal window 2124 therethrough that is oriented to oppose tissue-treating surface 2114 of jaw member 2110 in longitudinal alignment therewith. With body 2122 defining a cylindrical configuration, distal window 2124, defined therethrough, has an arcuate configuration with longitudinal sides of distal window 2124 defining an angle of equal to or less than 180 degrees relative to a longitudinal axis of body 2122.

A microwave antenna 2125 extends through body 2122 of jaw member 2120 such that at least a portion of the radiating section of microwave antenna 2125 is exposed through distal window 2124. Microwave antenna 2125 is adapted to connect to a source of microwave energy (not shown) such that, upon activation, microwave energy is radiated from microwave antenna 2125, through distal window 2124 and towards tissue-treating surface 2114 of jaw member 2110, while body 2122 of jaw member 2120 blocks microwave energy from radiating in other directions. In this manner, microwave energy is directed towards tissue-treating surface 2114 of jaw member 2110 and defines a radiating area, centered on a longitudinal axis of tissue-treating surface 2114 of jaw member 2110 and extending at an angle of equal to or less than 180 degrees (depending upon the angle of window 2124) relative to a longitudinal axis of body 2122. Microwave antenna 2125 may be configured to deliver various different microwave energies, e.g., from about 300 MHz to about 10,000 MHz, and, in embodiments, may be configured to provide a first energy and a second energy.

With reference to FIGS. 1, 6A-6D, and 7, a method of treating tissue in accordance with the present disclosure is described. Although detailed herein using end effector assembly 100 of surgical instrument 10 for treating liver tissue, it is understood that the method of treating tissue of the present disclosure may be performed with different end effector assemblies, instruments, or systems and/or may be performed on other tissue(s).

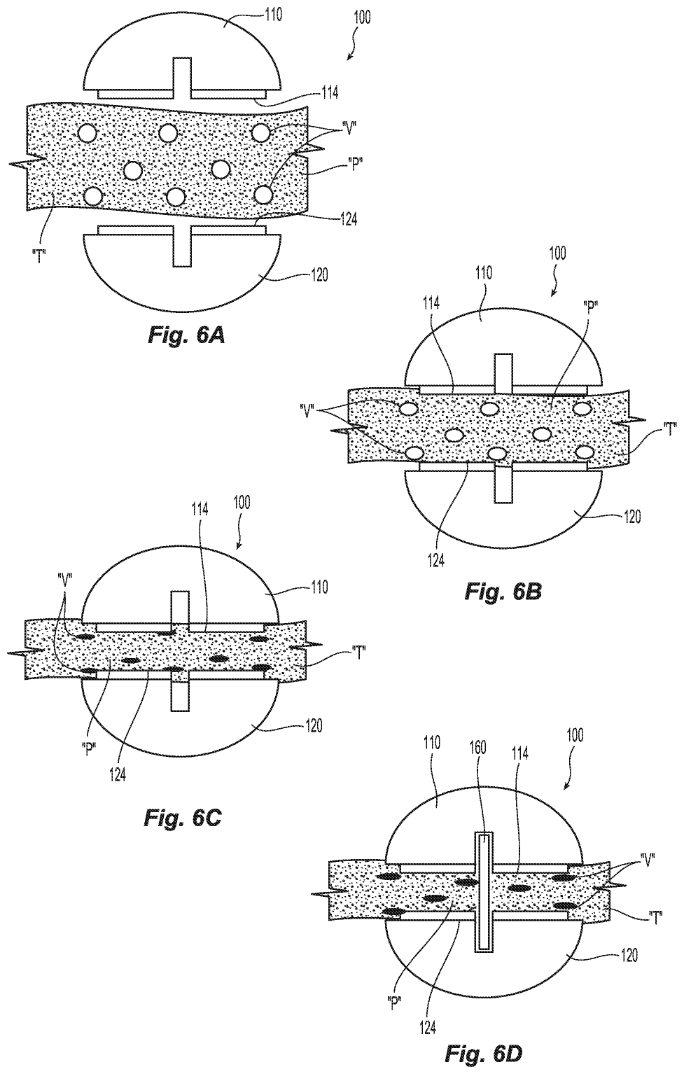

Referring initially to FIGS. 1 and 6A, with jaw members 110, 120 in the spaced-apart position, end effector assembly 100 is positioned about liver tissue “T” to be treated such that liver tissue “T” is disposed between tissue-treating surfaces 114, 124 of jaw members 110, 120, respectively, as illustrated in FIG. 6A. Liver tissue “T” includes parenchyma “P” and large vessels “V” extending through parenchyma “P.”

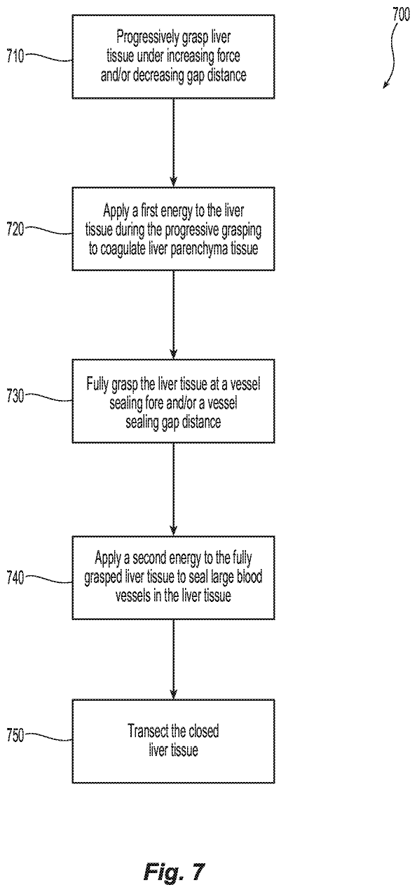

With additional reference to FIGS. 6B and 7, jaw members 110, 120 are progressively approximated relative to one another, e.g., via moving movable handle 40 relative to fixed handle 50 from the initial position towards the depressed position, to grasp liver tissue “T” between tissue-treating surfaces 114, 124 of jaw members 110, 120, respectively, as illustrated in FIG. 6B and step 710 (FIG. 7). As jaw members 110, 120 are progressively approximated, the closure force applied to liver tissue “T” is increased and/or the gap distance between jaw members 110, 120 is decreased, as indicated in step 710 (FIG. 7) such that liver tissue “T” and, more specifically, parenchyma “P” thereof is crushed down.

Continuing with reference to FIG. 6B, and referring to step 720 (FIG. 7), as jaw members 110, 120 are progressively approximated about liver tissue “T” to crush down parenchyma “P,” a first energy may be applied to tissue-treating surfaces 114, 124 of jaw members 110, 120 and conducted through liver tissue “T” grasped therebetween. The first energy may be applied by the electrosurgical generator “G” in response to activation of first activation switch 80 (see FIG. 1). More specifically, the first energy may be a standard bipolar energy supplied to tissue-treating surfaces 114, 124 of jaw members 110, 120 and conducted through liver tissue “T” to coagulate and permanently close parenchyma “P” as parenchyma “P” is crushed down by the approximating jaw members 110, 120. The first energy may be supplied continuously throughout the progressive approximation of jaw members 110, 120, intermittently throughout the progressive approximation of jaw members 110, 120, at stop points wherein jaw members 110, 120 are momentarily held stationary before continuing the progressive approximation, or in any other suitable manner.

With reference to FIG. 6C and step 730 (FIG. 7), jaw members 110, 120 are progressively approximated, as noted above, and eventually reach a fully grasped position, wherein jaw members 110, 120 apply vessel-sealing forces (constant forces or forces varying within a vessel-sealing force range) to liver tissue “T” grasped therebetween and/or define vessel-sealing gap distances (constant distances or distances varying within a vessel-sealing gap distance range) between tissue-treating surfaces 114, 124, respectively. “Vessel-sealing forces” and “vessel-sealing gap distances” as utilized herein refer to forces and gap distances, respectively, suitable for enabling vessel-sealing upon application of vessel-sealing energy. As appreciated, depending upon the particular settings, algorithm, etc. of vessel-sealing energy and/or the configuration of jaw members 110, 120, the vessel-sealing forces and vessel-sealing gap distances may vary. In embodiments, the vessel-sealing forces may range from about 3 kg/cm² to about 16 kg/cm², although other force ranges are also contemplated. In embodiments, the vessel-sealing gap distances may be range from about 0.001 inches to about 0.006 inches, although other gap distance ranges are also contemplated.

Continuing with reference to FIG. 6C, and referring to step 740 (FIG. 7), with jaw members 110, 120 grasping liver tissue “T” between tissue-treating surfaces 114, 124 under vessel-sealing forces and/or defining vessel-sealing gap distances, a second, vessel-sealing energy may be applied to tissue-treating surfaces 114, 124 of jaw members 110, 120 and conducted through liver tissue “T” grasped therebetween. The second, vessel-sealing energy may be applied by the electrosurgical generator “G” in response to further depression of movable handle 40 towards fixed handle 50 to activate the in-line second activation switch 90 (see FIG. 1). The vessel-sealing energy is an advanced bipolar energy that may include an energy-delivery algorithm with varying energy delivery, feedback-based control, and/or other features to facilitate vessel-sealing as opposed to simply coagulating tissue. The vessel-sealing energy is suitable for permanently closing large vessels “V” extending through the through parenchyma “P” of the liver tissue “T.”

Referring to FIG. 6D and step 750 (FIG. 7), once parenchyma “P” and vessels “V” are permanently closed, e.g., via coagulation using the first energy (FIG. 6B, step 720 (FIG. 7)) and vessel-sealing using the second energy (FIG. 6C, step 740 (FIG. 7)), respectively, knife 160 may be advanced between jaw members 110, 120 to cut the closed liver tissue “T.” Knife 160 may be advanced via actuation of trigger 62 of trigger assembly 60 (FIG. 1).

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A method of treating liver tissue, comprising:
progressively grasping liver tissue;
applying a first energy to the grasped liver tissue during the progressive grasping to permanently close parenchyma of the liver tissue;
fully grasping the liver tissue; and
applying a second energy to the fully grasped liver tissue to permanently close large vessels of the fully grasped liver tissue.

2. The method according to claim 1, wherein applying the first energy includes coagulating the parenchyma to permanently close the parenchyma.

3. The method according to claim 1, wherein the first energy is standard bipolar energy.

4. The method according to claim 1, wherein applying the second energy includes sealing the vessels to permanently close the vessels.

5. The method according to claim 1, wherein the second energy is a vessel-sealing energy.

6. The method according to claim 1, wherein fully grasping the liver tissue includes grasping the liver tissue with vessel-sealing forces.

7. The method according to claim 1, wherein fully grasping the liver tissue includes grasping the liver tissue between tissue-treating surfaces defining vessel-sealing gap distances therebetween.

8. The method according to claim 1, wherein fully grasping the liver tissue include:
grasping the liver tissue with vessel-sealing forces; and
grasping the liver tissue between tissue-treating surfaces defining vessel-sealing gap distances therebetween.

9. The method according to claim 1, wherein applying the first energy includes one of: continuously applying the first energy during the progressive grasping, intermittently applying the first energy during the progressive grasping, or applying the first energy at one or more stop points during the progressive grasping.

10. The method according to claim 1, further comprising cutting the liver tissue.

11. A method of treating liver tissue, comprising:
progressively approximating first and second jaw members to grasp liver tissue between respective first and second tissue-treating surfaces of the first and second jaw members;
conducting a first energy between the first and second tissue-treating surfaces and through the grasped liver tissue to permanently close parenchyma of the grasped living tissue as the first and second jaw members are progressively approximated;
further approximating the first and second jaw members to fully grasp the liver tissue between the first and second tissue-treating surfaces; and
conducting a second energy between the first and second tissue-treating surfaces and through the fully grasped liver tissue to permanently close large vessels of the fully grasped liver tissue.

12. The method according to claim 11, wherein conducting the first energy includes coagulating the parenchyma to permanently close the parenchyma.

13. The method according to claim 11, wherein the first energy is standard bipolar energy.

14. The method according to claim 11, wherein conducting the second energy includes sealing the vessels to permanently close the vessels.

15. The method according to claim 11, wherein the second energy is a vessel-sealing energy.

16. The method according to claim 11, wherein fully grasping the liver tissue includes grasping the liver tissue with vessel-sealing forces.

17. The method according to claim 11, wherein fully grasping the liver tissue includes grasping the liver tissue with vessel-sealing gap distances defined between the first and second tissue-treating surfaces.

18. The method according to claim 11, wherein fully grasping the liver tissue include:
grasping the liver tissue with vessel-sealing forces; and
grasping the liver tissue with vessel-sealing gap distances defined between the first and second tissue-treating surfaces.

19. The method according to claim 11, wherein conducting the first energy includes one of: continuously conducting the first energy during the progressive approximating, intermittently conducting the first energy during the progressive approximating, or conducting the first energy at one or more stop points during the progressive approximating.

20. The method according to claim 11, further comprising advancing a knife between the first and second jaw members to cut the liver tissue.
